(19) United States
(12) Patent Application Publication
(10) Pub. No.: US 2022/0304654 A1
(43) Pub. Date: Sep. 29, 2022

(54) ARTIFICIAL INTELLIGENCE FOR ASSESSMENT OF VOLUME STATUS USING ULTRASOUND

(71) Applicant: Gaurang Nandkishor Vaidya, Lexington, KY (US)

(72) Inventor: Gaurang Nandkishor Vaidya, Lexington, KY (US)

(21) Appl. No.: 17/717,122

(22) Filed: Apr. 10, 2022

(51) Int. Cl. A61B 8/12; A61B 8/08

(52) U.S. Cl. CPC A61B 8/12; A61B 8/0833; A61B 34/20

(57) ABSTRACT

The present invention comprises a novel method to utilize image tracking technology and artificial intelligence to make automatic measurements of the systemic vein lumen diameter and using calculations made to estimate a patient's volume status and cardiac ventricular function. In this technique, an ultrasound machine is used to measure the diameter of the systemic vein lumen and an image processing unit, such as a computing device, endowed with image recognition and tracking technologies through machine-learning, is used to measure the respiratory variation in the lumen diameter and circumference. Artificial intelligence technology is thereafter utilized to identify the maximum and minimum diameters/circumference and various calculations are made using the measured diameters, such as diameter variation percentage which is the difference between the maximum and minimum diameter divided by the maximum diameter and expressed as a percentage. Artificial intelligence is also used to identify whether a complete approximation of the vein diameter into 0 millimeters occurred with deep breathing and/or sniff. The above information is used to estimate the patient's volume and cardiac function status.

### FIELD OF INVENTION

The present invention is related to the use of artificial intelligence and image tracking technologies to measure systemic vein diameter variation with respiration and utilization of this information to ascertain a person's volume status, intracardiac pressure and cardiac function. More particularly, the invention relates to the use of portable imaging technology to assist in daily bedside patient-care decisions.

### BACKGROUND

Assessment of a patient's volume status is paramount in making important decisions related to the care of all patients on a regular basis. An accurate estimation, especially a diagnosis of excess water within the vascular system, can guide decisions related to use of diuretic medications and dialysis. It can also indicate a diagnosis of acutely decompensated heart failure. In addition, this is also necessary in multiple other conditions including kidney failure, high-output states such as septic shock, pulmonary hypertension, and endocrine disorders.

Invasive assessment of volume status using right heart catheterization (RHC) is the current gold standard for an accurate estimation. The procedure involves an invasive access into a systemic vein such as the internal jugular vein, followed by an insertion of a catheter into the right-sided chambers of the heart and lungs to measure the pressures directly. This however is an invasive and time-consuming procedure, only performed by credentialed physicians in well-equipped hospitals and not feasible for daily bedside use. The RHC procedure also has the inherent risks of bleeding, injury to the heart and other structures, as well as introduction of infection every time the procedure is performed. Medical students and other healthcare trainees are taught the inspection of the jugular vein on physical examination as a non-invasive alternative for volume status assessment. This inspection involves an assessment of the level of pulsation of the internal jugular vein in the neck of the patient. However, this relies on the jugular vein being superficial in the neck and factors which may obstruct the view of the vein, such as a patient's body habitus, often makes the assessment unreliable or unobtainable.

Similarly, patient's heart function can be assessed using an echocardiogram which, however, can only be obtained accurately by trained individuals committing multiple months of dedicated training. There is a shortage of trained sonographers and again represents a technique that is not available for all healthcare workers lacking dedicated training to perform an echocardiogram.

In this scenario, an ultrasound assessment of the systemic veins such as the inferior vena cava or internal jugular vein is employed by healthcare workers for volume estimation. This involves manual measurement of vein diameter variation with respiration. A larger vein diameter and a less dynamic respiratory variation in the diameter is indicative of excess vascular fluid content. A non-limiting example is provided in the detailed description where the ultrasound assessment of vein diameter variation can be used for cardiac function estimation.

The ultrasound assessment currently relies on manual measurements performed by the individual through “eyeballing”. This can easily introduce errors related to identification of the true vein wall and the angle of measurement. Finer errors in manual measurement can introduce significant inaccuracies in estimation. A need exists to automate the measurements to improve the accuracy. Automatic measurement can also reduce the learning-curve related to adoption of ultrasound technology at bedside.

### BRIEF SUMMARY OF THE EMBODIMENT

The present invention comprises a novel method to utilize image tracking technology to track the pixels which represent the walls of the vein in an image or a video obtained using an ultrasound machine. Through machine-learning, a computing device will be trained to identify and track vein walls. Artificial intelligence will make multiple measurements of the perpendicular distance between opposing walls of the vein throughout a respiratory cycle, using the tracked pixels.

The technology will then identify the correct maximum and minimum diameter from the multiple measurements made and calculate the maximum and minimum circumferences and cross-sectional areas of the vein per respiratory cycle. It will also calculate the volume of the vein per unit height, using the maximum and minimum areas of the vein.

The above information is used to estimate the patient's volume status, cardiac hemodynamic pressure and cardiac function.

### BRIEF DESCRIPTION OF THE DRAWINGS

Some embodiments of the present invention are illustrated as an example and are not limited by the figures of the accompanying drawings, in which like references may indicate similar elements and in which:

FIG. 1: FIG. 1 illustrates a non-limiting example of multiple measurements of the internal jugular vein diameter made by image tracking of an ultrasound M-mode image. Artificial intelligence identified the maximum (yellow line, marked #1) and the minimum diameter (blue line, marked #2) during one respiratory cycle.

FIG. 2: FIG. 2 illustrates a non-limiting example of complete anteroposterior collapse of the internal jugular vein diameter on sniff (A) as identified by artificial intelligence.

FIG. 3: FIG. 3 illustrates a non-limiting example of a computing device capable of computing internal jugular vein dimensions to display estimated intra-cardiac pressure. Maximum (A) and minimum (B) vein diameters are entered automatically and the estimated pressure is displayed (C).

### DETAILED DESCRIPTION OF THE EMBODIMENT

In the following detailed description, references are made to the accompanying drawings in which are shown the illustrations in how the embodiments may be practiced. It is to be understood that other embodiments may be utilized, with or without structural, procedural or logical changes, without departing from the scope. Therefore, the following description is not to be taken in a restricted or all-inclusive sense, and the scope of embodiments is defined by the appended claims and their equivalents. Moreover, the order of description of various procedures below should not be construed to imply that the procedures are order-dependent.

The description may use perspective-based descriptions such as up/down, back/front, supine/erect, anterior/posterior and top/bottom. Such descriptions are merely used to facilitate the discussion and are not intended to restrict the application of disclosed embodiments.

For the purposes of the description, a phrase in the form “A/B” or in the form “A and/or B” means (A), (B), or (A and B). For the purposes of the description, a phrase in the form “at least one of A and B” means (A), (B) or (A and B). The description may use the terms “embodiment” or “embodiments,” which may each refer to one or more of the same or different embodiments.

In the description, various apparatuses are described for use to carry out the various methods within the claims. In the future, a device may be endowed with the imaging and computing ability to perform all or various individual functions together and such a device may be employed to carry out the various methods as described below.

Embodiments described below provide methods to assess a subject's volume status, intra-cardiac pressures and cardiac function using an ultrasound of the systemic veins. Examples of systemic veins include inferior vena cava, internal jugular vein, subclavian vein and femoral vein. These ultrasound assessments have been correlated with direct intra-cardiac pressure measurement and cardiac function evaluation through gold-standard right heart catheterization technique.

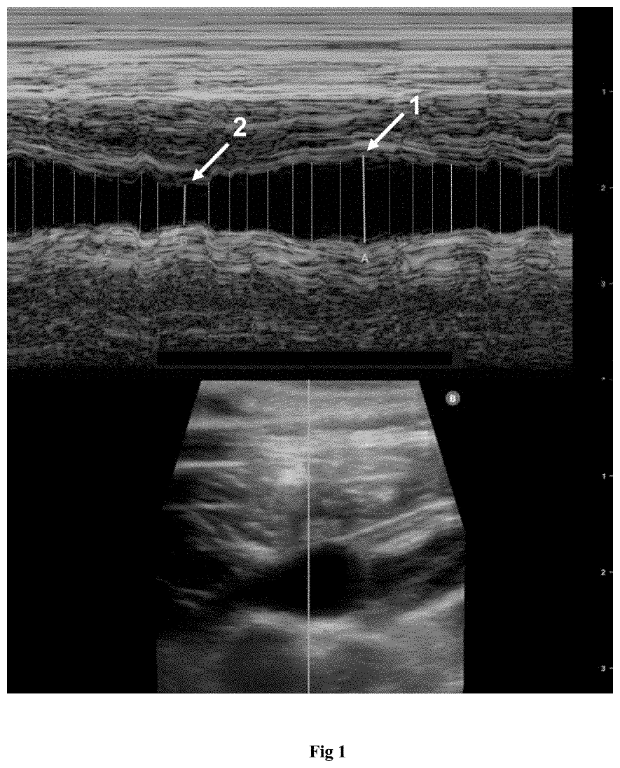

The methods involve acquisition of ultrasound imaging data of a suitable systemic vein. In some embodiment, images are obtained from the right or left internal jugular vein (IJV) in the subject. In the embodiment, as the individual breathes, the respiratory variation in the IJV dimensions is imaged. In the embodiment, a computing and processing device is trained through machine-learning to identify IJV within the ultrasound image/video. The images are thereafter processed by image recognition and tracking technology to identify pixels representing the opposing walls of the IV vein and tracked across time (FIG. 1). In the embodiment, artificial intelligence (AI) thereafter makes innumerable measurements of the distance between corresponding pixels from opposing IJV vein walls, representing the distances between the IJV walls at any given point in time (FIG. 1). In the embodiment, AI thereafter identifies the maximum and minimum IN lumen diameter, circumference and cross-sectional area. Lumen diameter, circumference and cross-sectional area are hereafter noted with an abbreviation-DCA. In the embodiment, AI calculates the difference between the maximum and minimum DCA of the IJV lumen and/or percentage variation in the IJV DCA over a time span, for instance the time span of one full inspiration and expiration cycle of respiration. In some embodiment, the AI also uses the diameter to calculate the maximum and minimum circumferences and cross-sectional areas of the vein per respiratory cycle as noted below. The AI will also calculate the volume of the vein per unit height, using the maximum and minimum areas of the vein as noted below:

Maximum circumference (MxC)=π×Maximum diameter
Minimum circumference (MnC)=π×Minimum diameter
Maximum cross-sectional area (MxA)=(π/4)×(Maximum diameter)²
Minimum cross-sectional area (MnA)=(π/4)×(Minimum diameter)²
Maximum volume per unit height=MxA×1
Minimum volume per unit height=MnA×1

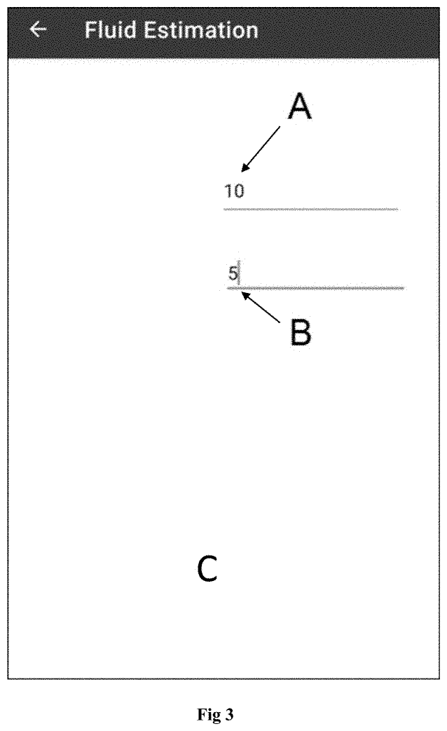

In the same embodiment, the obtained IJV dimension data is utilized to determine the subject's volume status and intra-cardiac pressure. The inventor describes an embodiment of a protocol to estimate volume status using IJV data as noted below. Intra-cardiac pressure includes both right and left intra-cardiac pressures. The data is thereafter displayed on a display device and stored for future use (FIG. 3).

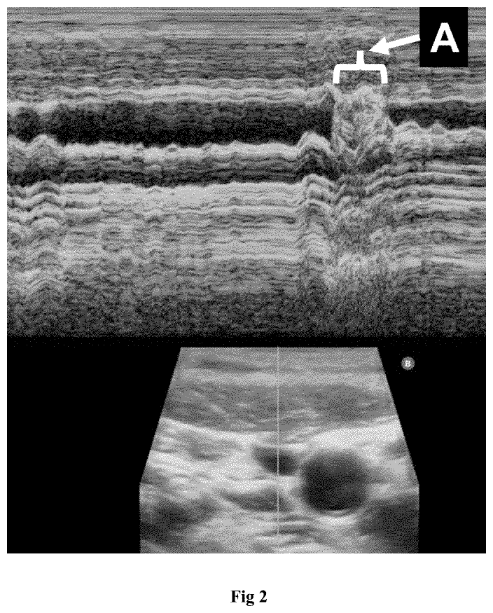

In other embodiments, the methods include the processing the IJV dimension data which further comprises of determination by the AI of the extent of IJV diameter diminution on deep/full inspiration and/or sniff maneuver by the subject (FIG. 2).

In another embodiment, similar data is obtained from the subclavian vein (SCV) or inferior vena cava (IVC) or femoral vein (FMV). The data from the SCV, IVC or FMV are processed similarly to estimate the volume status and/or intracardiac pressures.

The description below also provides a method to assess cardiac function through acquisition of ultrasound imaging data from systemic veins across the time span and the measurements and calculations made as described above, and thereafter determining the indicators of cardiac function from the data. The time span comprises for instance at least one respiratory cycle including an inspiration and an expiration. The indicators of cardiac function include an estimate of the cardiac output, cardiac index, right ventricular stroke work index and/or pulmonary artery pulsatility index. The estimated cardiac function is displayed on a display device and stored for future use.

The techniques may be implemented according to various embodiments described herein using an ultrasound device capable of imaging the systemic veins and have an ability to measure the diameter, for instance using M-mode technique within the ultrasound. The ultrasound device processing unit may be capable of tracking and measuring the diameter variation as occurring during the respiratory cycle, for instance using image recognition and image tracking technology. According to the embodiments, the images may then be displayed on a display monitor. In some embodiments, an AI contained in the processing unit will be capable of determining the maximum and minimum diameters and make calculations using the diameter measurements. The acquired dimension and respiratory variation data is provided to an image processing or computing device for determination of volume status, intra-cardiac pressures and/or cardiac function.

Assessment of a patient's volume status is a common daily assignment for clinicians. An accurate estimation of a patient's body water, especially an excess within the vascular system, is of paramount importance in the management of heart failure patients. Moreover, the utility of this assessment spans beyond cardiologists to also include internists, hospitalists, family medicine practitioners, nephrologists etc. Currently the only techniques feasible for daily bedside use and also widely practiced to provide a volume estimation is the physical examination of the jugular venous distension which can be inaccurate and unreliable based on a patient's body habitus and neck circumference. Ultrasound imaging the systemic veins such as inferior vena cava or internal jugular vein is feasible at bedside for volume status estimation. In patients with excess fluid in the vascular space, the veins have larger diameter and less profound respiratory variation in diameter. However, the acquisition of imaging data manually can be inaccurate. It has a potential to introduce errors due to a requirement of manual measurements of the diameter through “eye-balling” and identifying the vein wall and the maximum and minimum diameters. Manual measurements and calculations also increase the time to acquisition, making them inefficient and inaccurate for daily assessment. The gold standard for volume estimation is a right heart catheterization (RHC) which is not feasible for daily use, invasive, time-consuming and expensive.

Additionally, bedside assessment of cardiac function involves performing an ultrasound of the heart directly, a procedure called echocardiography. Performing an echocardiogram comprises a significant learning curve for most healthcare workers, requiring multiple months of dedicated training. There is also a shortage of sonographers trained in performing echocardiograms. This makes the technique not available or feasible for a majority of healthcare workers.

On the other hand, an automated imaging unit capable of tracking, measuring and calculating respiratory variation in vein diameter can provide an easy to adopt technology at bedside, with minimum training and more likelihood of widespread adoption. In addition to intracardiac pressure assessment, respiratory variation in systemic vein diameter can also provide an alternative strategy for assessing cardiac function as described below. Healthcare workers can use the ultrasound probe to image superficial systemic veins such as IV. The processing or computing unit of the ultrasound, endowed with the abilities as described in the endowment above and claims below, can process the images and perform automatic measurements and calculations accurately. Vital bedside data will be displayed to the healthcare workers for ease of use in daily practice.

Bedside ultrasound is possible using portable ultrasound machines widely available in all hospitals. Portable ultrasound probes are also available for commercial purchase by individual practitioners, which have the ability to connect with most smartphones and tablets. However, all ultrasound machines rely on manual measurements of the vein diameter variation, if used for this purpose. A portable ultrasound imaging and processing unit endowed with an ability to provide a truly objective and accurate bedside assessment of a subject's fluid status and cardiac function, with minimal learning curve for practitioners and with minimal to no discomfort to the patients, will be a crucial addition to daily practice. This technique could be vital, for instance, in the hands of primary care practitioners within community centers and outreach hospitals.

Thus, enclosed herein, in various embodiments, are methods to utilize ultrasound image tracking technology and artificial intelligence to make an accurate estimation of the subject's volume status and cardiac function. The accuracy of the obtained IJV, SCV and IVC dimension and respiratory variation data in estimating intracardiac pressure and cardiac function was demonstrated in correlation with simultaneously performed right heart catheterization (RHC) as described below.

In a specific, non-limiting embodiment, 72 patients scheduled to undergo RHC within the Jewish hospital and University of Louisville hospital in Louisville were enrolled in a prospective study. The study protocol was reviewed and approved by the institutional review board of the University of Louisville. All patients included in the study signed an informed consent. The inclusion criteria included: spontaneously breathing adults (age >18 years) and able to consent. Patients with orthotopic heart transplant (OHT) or left ventricular assist device (LVAD) were also eligible for enrollment. Exclusion criteria included: known occlusion of IJV, superior vena cava obstruction/compression or severe tricuspid regurgitation.

For the purpose of the study, patients were educated about the study procedures including the sniff maneuver. For standardization of patient positioning within this specific and non-limiting embodiment, patients were then positioned supine at 0 degrees with their head in neutral position and breathing restfully. Next, the right sternocleidomastoid muscle was identified and the right IJV was imaged at the apex of the triangle formed by the sternal and clavicular heads of the muscle. If the patient had an indwelling intravenous catheter or an implanted device, such as a pacemaker, on one side of the neck or chest wall then the left IJV was used. Similarly, the SCV was imaged at the junction of the lateral third and the middle third of the right clavicle.

For this particular non-limiting embodiment, a portable ultrasound system-Sonosite (Bothell, Wash.) was used for imaging purposes. Using M-mode technique of the ultrasound, the maximum and minimum anteroposterior diameters of IJV/SCV at the above-mentioned landmarks, were noted during normal breathing, without applying any external pressure.

The respiratory variation in diameter (RVD) was calculated as [(maximum diameter−minimum diameter)/maximum diameter] and expressed as percent. The patients were then asked to sniff forcefully. The anteroposterior diameter collapsibility was assessed on sniff maneuver. The first 10 imaging acquisitions were timed.

The patients then underwent right heart catheterization within 1 h of the ultrasound assessment and right atrial (RA) pressure, right ventricular (RV) pressure, pulmonary artery (PA) pressure and pulmonary capillary wedge pressure (PCWP) were recorded.

IBM SPSS (version 24.0, SPSS Corp, Chicago, Ill., USA) was used for statistical analysis. Qualitative data is presented as frequencies and quantitative data as mean f standard deviation. Categorical variables and continuous variables were analyzed using Chi-square test, and Student's t-test respectively. The correlation of imaging parameters to invasive RA pressure measurement was assessed using linear regression. Receiver operating curve (ROC) analysis was performed to determine the sensitivity and specificity of imaging parameters in estimation of right atrial pressure with the invasive RA pressure as the gold standard. A two-sided p-value <0.05 was considered significant.

Total of 72 patients were enrolled in the study with mean age 61±14 years, and mean BSA 1.9±0.2m2. None of the patients were ventilator dependent or on intravenous inotropic/vasoactive agents. Echocardiography data was available in 81% of patients within one month of enrollment and the mean LVEF was 45% (10-75%). Forty percent of patients had BMI ≥30 kg/m2 (table 1A).

TABLE 1A
Baseline characteristics of the patient population

| Variable | Frequency/Mean | |
|---|---|---|
| Male | 61% | |
| Age (years) | 60.8 ± 14.0 | (21-85) |
| Body surface area (m²) | 1.9 ± 0.2 | (1.3-17.4) |
| Body mass index (kg/m²) | 30.0 ± 6.5 | (17.4-48.1) |
| Systolic blood pressure (mmHg) | 125.4 ± 24.0 | (54-196) |
| Heart rate (beats/min) | 75.8 ± 15.5 | (52-110) |
| Atrial fibrillation | 9% | |
| Trace/mild tricuspid regurgitation | 89% | |
| LV ejection fraction (%) | 45.2 ± 20.0 | (10-75) |
| Recurrent catheterization | 14% | |
| Serum Creatinine (mg/dL) | 1.47 ± 1.46 | (0.39-10.56) |
| Blood urea nitrogen/creatinine ratio | 17.31 ± 6.6 | (4.0-37.2) |
| Serum bicarbonate (mg/dL) | 25.8 ± 3.5 | (11.3-34.0) |

Normal LVEF was defined as ≥52% in males and ≥54% in females based on American Society of Echocardiography guidelines. Half the patients with available data had normal ejection fraction and 42% had EF≤35%. The cohort included 6 (8%) OHT recipients and 4 (6%) patient with LVAD implantation.

Image acquisition required <5 minutes per patient as assessed in the first 10 patients. UV could be imaged in all patients irrespective of their body mass index (BMI).

Right heart catheterization (RHC) findings are described in table 11B; 35% of patients had RA pressure ≥10 mmHg and 31% had at least moderate pulmonary hypertension. All patients with maximum IN diameter <0.5 cm had RA pressure <10 mmHg (12 patients, 17%). Similar findings were noted with RVD in IJV>50% (16 patients, 22%).

TABLE 1B
Right heart catheterization findings

| Variable (mmHg) | Mean | |
|---|---|---|
| Right atrial pressure (mmHg) | 8.3 ± 5.3 | (0-20) |
| Pulmonary systolic pressure (mmHg) | 44.7 ± 20.2 | (16-120) |
| Pulmonary mean pressure (mmHg) | 28.8 ± 13.0 | (10-74) |
| Pulmonary capillary wedge pressure (mmHg) | 15.5 ± 9.3 | (4-48) |

Patients with elevated RA pressure (≥10 mmHg) showed less RVD in IJV with respiration in resting condition (14 vs. 40%, p=0.01). They also had larger maximum IJV diameter (p=0.01) [table 2].

TABLE 2
Elevated RA pressure and IJV diameter variation on respiration

| | RA pressure ≥ | RA pressure < | |
|---|---|---|---|
| | 10 mmHg | 10 mmHg | P-value |
| Maximum IJV diameter | 1.0 ± 0.2 | 0.7 ± 0.3 | 0.001 |
| (cm) | | | |
| Percent IJV diameter | 14% | 40% | 0.001 |
| variation | | | |
| Complete IJV AP | 16% | 66% | 0.001 |
| collapsibility on sniff | | | |

Complete collapsibility of IJV anteroposterior diameter with sniff maneuver was associated with significantly lower RA (5.2 vs 11.3 mmHg, p=0.001) and PCWP pressures (12.2 vs 18.5 mmHg, p=0.004) [table 3].

TABLE 3
IJV collapsibility on sniff and the
right heart catheterization findings

| | IJV collapsible | Not collapsible | P-value |
|---|---|---|---|
| Right atrial pressure | 5.2 ± 2.8 | 11.3 ± 5.4 | 0.001 |
| (mmHg) | | | |
| Pulmonary systolic | 36.2 ± 12.7 | 52.7 ± 22.7 | 0.001 |
| pressure (mmHg) | | | |
| Pulmonary mean | 23.2 ± 8.2 | 34.1 ± 14.4 | 0.001 |
| pressure (mmHg) | | | |
| Pulmonary capillary | 12.2 ± 7.3 | 18.5 ± 10.1 | 0.004 |
| wedge pressure (mmHg) | | | |

Sensitivity and specificity analysis were performed to assess accuracy of IJV ultrasound in estimating high RA pressure (table 4). For RA pressure ≥10 mmHg, lack of IJV collapsibility with sniff had a sensitivity of 84% and specificity of 66%. A maximum IJV diameter ≥1 cm and respiratory variation <50% had a sensitivity of 60% and specificity of 80% with ROC area 0.694 for RA pressure ≥10 mmHg (table 4). Similarly, a maximum IJV diameter ≥1 cm and lack of complete IJV collapsibility with sniff had a sensitivity and specificity of 56% and 83% respectively.

TABLE 4
Sensitivity and specificity of various IJV findings
in predicting RA pressure ≥ 10 mmHg.

| | Sensitivity | Specificity | ROC AUC |
|---|---|---|---|
| Maximum IJV diameter ≥ 1 cm | 60% | 72% | 0.662 |
| No IJV collapsibility with sniff | 84% | 66% | 0.750 |
| Maximum IJV diameter ≥ 1 cm + | 56% | 83% | 0.708 |
| no collapsibility | | | |
| Maximum IJV diameter ≥ 1 cm + | 60% | 80% | 0.694 |
| percent variation < 50% on normal | | | |
| respiration | | | |

Among the subgroup of patients with EF≤35% (30 patients), the percent diameter variation continued to have positive correlation with RA pressures (R=0.66, p=0.001). Similarly, for patients with mean pulmonary pressure ≥35 mmHg, the positive correlation between percent variation and RA pressure was maintained (R=0.66 for IJV, p=0.001). Based on the above data, an algorithm was constructed to estimate RA pressure at bedside with considerable certainty.

There was a positive correlation between IJV diameter difference and pulmonary artery pulsatility index (PAPi) (p=0.012). The PAPi is calculated as the difference of pulmonary systolic and diastolic pressure divided by RA pressure as measured on RHC. Correlation was also detected with right ventricular stroke work index (p=0.04), which is calculated as 0.0136×stroke volume index×(mean pulmonary artery pressure-RA pressure). Stroke volume index was calculated as cardiac index by the Fick method divided by heart rate. Both PAPi and RVSWI are considered indicators of right ventricular function.

Similar results were noted with SCV (tables 5 and 6).

TABLE 5
Elevated RA pressure and SCV diameter variation on respiration

| | RA pressure ≥ | RA pressure < | |
|---|---|---|---|
| | 10 mmHg | 10 mmHg | P-value |
| Maximum SCV | 0.8 ± 0.2 | 0.6 ± 0.3 | 0.270 |
| diameter (cm) | | | |
| Percent SCV | 24% | 45% | 0.011 |
| diameter variation | | | |
| Complete SCV AP | 25% | 57% | 0.012 |
| collapsibility on sniff | | | |

TABLE 6
SCV collapsibility on sniff and the
right heart catheterization findings

| | SCV collapsible | Not collapsible | P-value |
|---|---|---|---|
| Right atrial pressure | 6.2 ± 4.6 | 10.5 ± 5.4 | 0.001 |
| (mmHg) | | | |
| Pulmonary systolic | 39.3 ± 19.3 | 49.9 ± 20.8 | 0.037 |
| pressure (mmHg) | | | |
| Pulmonary mean | 24.6 ± 11.2 | 32.7 ± 14.0 | 0.013 |
| pressure (mmHg) | | | |
| Pulmonary capillary | 11.5 ± 6.1 | 19.2 ± 10.3 | 0.001 |
| wedge pressure (mmHg) | | | |

The study reports, a strong positive correlation of internal jugular vein and subclavian vein diameters as well as their collapsibility, as assessed with bedside ultrasound, with invasive right heart catheterization. The study cohort represents a real-world population of patients including patients with heart failure, pulmonary hypertension, LVAD and heart transplant. The study demonstrated 1) A significant positive correlation between the vein diameters and RA pressure, 2) Less respiratory variation and larger vein diameters with elevated RA pressure, and 3) Lack of IJV collapsibility as a highly sensitive marker for higher RA pressure. 4) Correlation of the UV diameter variation with right ventricular function parameters. The study results have direct application in day-to-day care of patients.

The study however relied on manual measurements from the inventor, which can introduce human errors from identifying the true maximum and minimum diameters and accurately identifying the walls of the vein by inspection with the eyes. Finer errors can introduce a large error in calculation of the sub-centimeter vein lumens as noted in the tables above (tables 2 and 5).

Therefore, an embodiment is described in the application and claims are made of a processing or computing unit capable of tracking vein walls and making accurate measurements of the diameter of the vein lumen (FIG. 1). The endowment is capable of automatically identifying the maximum and minimum diameter and the variation occurring in the diameter with respiration and calculating the circumference, cross-sectional area and volume of the vein lumen. The endowment is also capable of identifying a complete approximation of opposing vein walls on sniff maneuver or deep/full inspiration (FIG. 2). The endowment thereafter is also capable of processing the measurements to obtain volume status, intracardiac pressure and/or cardiac function using established protocols and display the obtained data on a display device (FIG. 3).

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein.

What is claimed:

1. A method for assessing a subject's volume status and intra-cardiac pressures comprising: acquisition of ultrasound imaging data of the systemic veins in the subject; utilization of technology capable of making automatic measurements of the vein lumen dimensions over a time span; processing the vein dimension data using artificial intelligence and determining the volume status and/or intra-cardiac pressure from the vein dimension data.

2. The method of claim 1, further comprising: a technology capable of automatically tracking and measuring the vein dimension variation over a time span.

3. The method of claim 1, wherein the time span comprises of one full inspiration and expiration cycle of respiration.

4. The method of claim 1, wherein the measurement of vein dimension data comprises measurement of the diameter, surface area and circumference of the vein lumen.

5. The method of claim 1, wherein the systemic veins comprise internal jugular vein, subclavian vein, inferior vena cava or femoral vein.

6. The method of claim 1, further comprising: artificial intelligence identifying maximum and minimum dimensions during respiration over a time span and identifying separately whether complete approximation of vein walls occurred when the subject was asked to take a deep inspiration or sniff.

7. The method of claim 1, wherein processing the vein dimension and respiratory data comprises calculating the difference between the maximum and minimum dimensions of the vein lumen and/or percentage variation in the vein dimensions over a time span

8. The method of claim 1, further comprising: determination of intra-cardiac pressure through processing of vein dimension and respiratory data by a computing device.

9. The method of claim 1, further comprising of recording and displaying of the volume status and intracardiac pressure on a display capable device.

10. The method of claim 7, wherein percentage variation in the vein dimensions comprises the calculation of the difference between the maximum and minimum vein dimensions divided by the corresponding maximum vein dimension.

11. A method to evaluate heart function of a subject, the method comprising: acquisition of ultrasound imaging data of a systemic vein in the subject: utilization of image recognition and tracking technology to automatically measure vein dimension variation over a time span; utilization of artificial intelligence to identify maximum and minimum dimensions; processing the vein dimension data and determining the indicators of cardiac function through calculations made by a computing device.

12. The method of claim 11, wherein the measurement of vein dimension data comprises measurement of the maximum and minimum diameter, surface area and circumference of the vein lumen.

13. The method of claim 11, wherein processing the vein dimension data comprises calculating the difference between the maximum and minimum dimensions of the vein lumen and/or percentage variation in the vein dimensions over a time span

14. The method of claim 11, wherein the time span is at least one respiratory cycle comprising an inspiration and an expiration.

15. The method of claim 11, wherein the indicator of heart function is an estimate of the cardiac output, cardiac index, the right ventricular stroke work index and/or pulmonary artery pulsatility index.

16. The method of claim 11, further comprising of recording and displaying the estimated cardiac function on a display device.

17. A system for executing the method of claims 1 and 11

18. The system of claim 17 which comprises: an ultrasound imaging and processing apparatus capable of capturing and processing imaging data

19. The method of claim 17 wherein, the processing of imaging data comprises a device capable of identifying and tracking vein dimensions, performing automatic measurements of vein dimensions and artificial intelligence to make decisions on identifying the maximum and minimum dimensions as well as computing capabilities to make required calculations to compute the volume status and heart function.

20. The method of claim 17, further comprising a device capable of displaying the volume status and heart function in a human-readable format.
